(19) United States
(12) Patent Application Publication
(10) Pub. No.: US 2026/0363912 A1
(43) Pub. Date: Nov. 19, 2026

(54) INTRAOCULAR FLUID DRAINAGE DEVICE FOR TREATING GLAUCOMA

(71) Applicant: Mark J. Gallardo, El Paso, TX (US)

(72) Inventor: Mark J. Gallardo, El Paso, TX (US)

(21) Appl. No.: 19/533,561

(22) Filed: Feb. 9, 2026

Related U.S. Application Data
(60) Provisional application No. 63/756,463, filed on Feb. 10, 2025.

(51) Int. Cl. A61F 9/007

(52) U.S. Cl. CPC A61F 9/00781

(57) ABSTRACT

An intraocular fluid drainage device is described. The device can include an input tube shunt. The input tube shunt can provide a fluid conduit between a distal end of the tube shunt, which is configured for insertion into an anterior chamber of an eye of a subject, and a proximal end of the tube shunt. The device can include a fluid receptacle and reservoir. This can include a fluid-expandable first compliant bag. The first compliant bag can include a first input port coupled to the proximal end of the tube shunt to receive intraocular fluid therefrom. The first compliant bag can include a first output port from which to discharge intraocular fluid, either into another compliant bag, or out from the reservoir and into a physiological space in the subject.

### CLAIM OF PRIORITY

This patent application claims the benefit of priority of Mark J. Gallardo U.S. Provisional Patent Application No. 63/756,463 entitled INTRAOCULAR FLUID DRAINAGE DEVICE FOR TREATING GLAUCOMA, filed Feb. 10, 2025 (Attorney Docket No. 5462.004PRV), which is hereby incorporated herein by reference in its entirety, and the benefit or priority of which is claimed.

### TECHNICAL FIELD

This document pertains generally, but not by way of limitation, to ophthalmic devices and methods of treatment, and more particularly, but not by way of limitation, to an intraocular fluid drainage device, such as for treating glaucoma.

### BACKGROUND

Glaucoma is an eye condition characterized by a progressive optic neuropathy with or without visual field loss. The only modifiable risk factor is the intraocular pressure (IOP), which can be achieved through several therapeutic options, including drops, laser surgery, and incisional surgery. IOP can be reduced by reducing aqueous humor production or facilitating aqueous outflow through the eye's natural drainage system or through one or more iatrogenically created shunts. Within incisional surgery, filtration surgery is one category of procedures that drains the pressure-inducing aqueous humor fluid from within the eye into extraocular tissues or reservoirs.

When aqueous humor is drained from the anterior chamber of the eye into the extraocular tissues, a bleb formation tissue reaction can occur, leading to a fluid reservoir referred to as a “bleb”. Commonly, blebs are surrounded by or encapsulated by a fibrotic capsule, which can impede diffusion of aqueous humor and eventual re-elevation of the IOP. Contrarily, thin blebs can also be associated with anti-fibrotic drugs administered during or after surgery, increasing the risk for low intraocular pressures and/or infections.

Ahmed U.S. Pat. No. 8,632,489 is directed to an implantable device having a porous device chamber into which aqueous humor fluid can be drained. In Ahmed, the aqueous humor fluid is drained via a tube shunt having a distal end inserted into an anterior chamber of the eye. A proximal end of the Ahmed tube shunt is attached to a valve that discharges aqueous fluid into the porous device chamber, from which the aqueous fluid can be then be discharged into the vasculature via pores in device chamber walls. Fibrotic tissue ingrowth into the pores can occur, which can make removal and explantation of the device challenging.

Ahmed U.S. Patent Pub. No. US2021/0275358A1 is directed to a similar single chamber device with microgrooves on an outer surface.

Baerveldt U.S. Pat. No. 8,920,357 is directed to a valveless implantable device that includes a flexible plate or section that is coupled to a drainage tube. The Baerveldt drainage tube is temporarily ligated upon insertion, but restores its patency over time, such that it serves as a flow restrictor that regulates outflow from the eye until a bleb forms around the plate.

### SUMMARY/OVERVIEW

An intraocular fluid drainage device is described. The device can include an input tube shunt. The input tube shunt can provide a fluid conduit between a distal end of the tube shunt, which is configured for insertion into an anterior chamber of an eye of a subject, and a proximal end of the tube shunt. The device can include a fluid receptacle and reservoir. This can include a fluid-expandable first compliant bag. The first compliant bag can include a first input port coupled to the proximal end of the tube shunt to receive intraocular fluid therefrom. The first compliant bag can include a first output port from which to discharge intraocular fluid, either into another compliant bag, or out from the reservoir and into a physiological space in the subject.

This Summary/Overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of portions of an intraocular fluid drainage device.

FIG. 2 is a flow chart illustrating generally portions of a method of using a device, such as shown in FIG. 1, for draining intraocular fluid from an anterior portion of an eye of a subject.

### DETAILED DESCRIPTION

An intraocular fluid drainage device is described. The device can include an input tube shunt. The input tube shunt can provide a fluid conduit between a distal end of the tube shunt, which is configured for insertion into an anterior chamber of an eye of a subject, and a proximal end of the tube shunt. The device can include a fluid receptacle and reservoir. This can include a fluid-expandable first compliant bag. The first compliant bag can include a first input port coupled to the proximal end of the tube shunt to receive intraocular fluid therefrom. The first compliant bag can include a first output port from which to discharge intraocular fluid, either into another compliant bag, or out from the reservoir and into a physiological space in the subject.

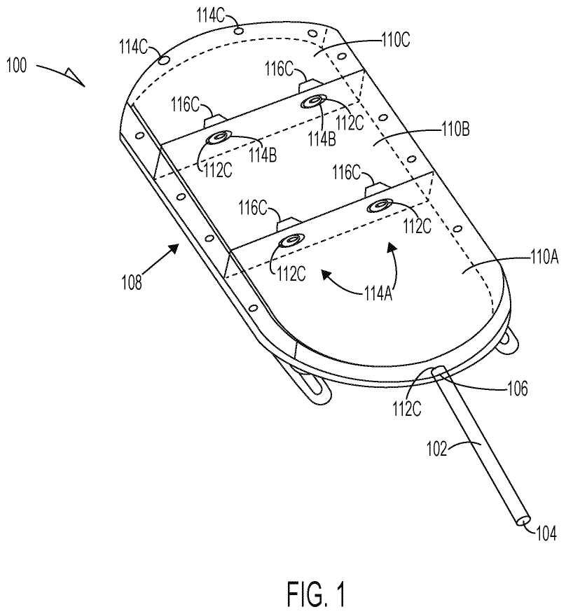

FIG. 1 shows an example of portions of an intraocular fluid drainage device 100. The drainage device 100 can include an input tube shunt 102. The tube shunt 102 can include a tube defining a coaxial longitudinal lumen extending therethrough for the length of the tube shunt 102. Via this longitudinal lumen, the tube shunt 102 can provide a fluid conduit between a distal end 104 of the tube shunt 102, which is configured for insertion into an anterior chamber of an eye of a subject, and a proximal end 106 of the tube shunt 102. The fluid conduit of the tube shunt 102 can allow for drainage of aqueous humor intraocular fluid from the anterior chamber of the eye of the subject, such as to help decrease the intraocular pressure (IOP) in the eye of the subject.

The drainage device 100 can also include a fluid receptacle and reservoir 108. The reservoir 108 can include a fluid-expandable first compliant bag 110, such as which can provide a fluid-expandable hollow chamber into which drains aqueous humor intraocular fluid from the anterior chamber of the eye of the subject, such as via the longitudinal lumen of the tube shunt 102. The first compliant bag 110A can include a first input port 112A that can be included in coupled to the proximal end 106 of the tube shunt 102 to receive intraocular fluid therefrom. The first compliant bag 110A can include at least one first output port 114A from which to discharge accumulated intraocular fluid, such as further explained herein.

The first output port 114A can include a valve 116A therethrough, such as through which fluid can pass when the valve 116A is in an open state, but through which fluid cannot pass when the valve 116A is in a closed state. In an example, the valve 116A can include a duckbill or other one-way valve that is responsive to an upstream pressure or that is responsive to an upstream-downstream pressure differential. For example, the valve 116A can be configured to automatically open to discharge intraocular fluid from the first compliant bag 110A when a first fluid pressure within the first compliant bag 110A has reached or exceeded a specified first threshold pressure, such as can be specified to be at least 15 mm Hg (e.g., specified at a selected one of 15, 16, 17, 18, 19, 20, 21, 22, or 23 mm Hg). The particular first threshold pressure value at which the valve 116A opens can be obtained by selecting an appropriate valve 116A having such a first threshold pressure valve-opening property, or it can be selected by using a programmable valve that can be programmed to select a particular valve-opening pressure from a variety of different available valve-opening pressures.

In an example, a fully expanded fluid volume of the first compliant bag 110A is between 0.05 milliliters and 0.40 milliliters, which can be selected based on an expected or actual intraocular fluid drainage rate to leave a sufficient period of time (e.g., between 2 days and 7 days) such as to promote controlled bleb formation about the reservoir 108, such that any resulting bleb is not too thick to inhibit transfer of intraocular fluid from the reservoir 108 and out to the vasculature to be returned to circulation via the vasculature. The fluid-expanding nature of the first compliant bag 110A, from its collapsed fluid volume of approximately zero milliliters to its fully expanded fluid volume is believed to help promote controlled bleb formation, such that any resulting bleb is not too thick to inhibit transfer of intraocular fluid from the reservoir 108 and out to the vasculature to be returned to circulation via the vasculature. Similarly, the fluid-impervious non-porous construction of the first compliant bag 110A (except for its one or more input ports 112A and its one or more output ports 114A that are defined in the non-porous material otherwise forming the first compliant bag 110A) also helps resist bleb ingrowth into the first compliant bag 110A, which is also believed to help promote controlled bleb formation, such that any resulting bleb is not too thick to inhibit transfer of intraocular fluid from the reservoir 108 and out to the vasculature to be returned to circulation via the vasculature.

In an example, the first output port 114A from the first compliant bag 110A can optionally provide a fluid pathway into a fluid-expandable second compliant bag 110B, which can also be included within the fluid receptacle and reservoir 108. The second compliant bag 110B can include at least one input port 112B included with or coupled to the first output port 114A of the first compliant bag 110A to receive intraocular fluid therefrom, The second compliant bag 110B can include at least one second output port 114B from which to discharge intraocular fluid that has accumulated in the second compliant bag 110B. The second output port 114B can include a second valve 116B. The second valve 116B can be configured to open to discharge intraocular fluid from the second compliant bag 110B when a second fluid pressure within the second compliant bag 110B has reached or exceeded a specified second threshold pressure, such as can be specified to be at least 12 mm Hg (e.g., specified at a selected one of 12, 13, 14, 15, 16, 17, 18, 19, 20, 21, 22, or 23 mm Hg). The particular second threshold pressure value at which the valve 116B opens can be obtained by selecting an appropriate valve 116B having such a second threshold pressure valve-opening property, or it can be selected by using a programmable valve that can be programmed to select a particular valve-opening pressure from a variety of different available valve-opening pressures. When the second threshold pressure valve opening pressure (e.g., 12 mm Hg) into the second compliant bag 110B is less than the first threshold pressure valve opening pressure (e.g., 15 mm Hg) into the first compliant bag 110A, a serial vacuum effect can be provided by the relatively downstream second compliant bag 110B, which, in turn, can help urge fluid into the first compliant bag 110A.

In an example, a fully expanded fluid volume of the second compliant bag 110B is between 0.05 milliliters and 0.04 milliliters, which can be selected based on an expected or actual intraocular fluid drainage rate from the second compliant bag 110B to leave a sufficient period of time (e.g., between 2 days and 7 days) to promote controlled bleb formation about the reservoir 108, such that any resulting bleb is not too thick to inhibit transfer of intraocular fluid from the reservoir 108 and out to the vasculature to be returned to circulation via the vasculature, as similarly explained above with respect to the first compliant bag 110A.

In an example, the second output port 114B from the second compliant bag 110B can optionally provide a fluid pathway into a fluid-expandable third compliant bag 110C, which can also be included within the fluid receptacle and reservoir 108. The third compliant bag 110C can include at least one input port 112C included with or coupled to the second output port 114B of the second compliant bag 110B to receive intraocular fluid therefrom, The third compliant bag 110C can include at least one third output port 114C from which to discharge intraocular fluid that has accumulated in the third compliant bag 110C. The third output port 114C can include a third valve 116C. The third valve 116C can be configured to open to discharge intraocular fluid from the third compliant bag 110C when a third fluid pressure within the third compliant bag 110C has reached or exceeded a specified third threshold pressure, such as can be specified to be at least 8 mm Hg (e.g., specified at a selected one of 8, 9, 10, 11, 12, 13, 14, 15, 16, 17, 18, 19, 20, 21, 22, or 23 mm Hg). The particular third threshold pressure value at which the third valve 116C opens can be obtained by selecting an appropriate valve 116C having such a third threshold pressure valve-opening property, or it can be selected by using a programmable valve that can be programmed to select a particular valve-opening pressure from a variety of different available valve-opening pressures. When the third threshold pressure valve opening pressure (e.g., 8 mm Hg) into the third compliant bag 110C is less than the second threshold pressure valve opening pressure (e.g., 12 mm Hg) into the second compliant bag 110B, a serial vacuum effect can be provided by the relatively downstream third compliant bag 110C, which, in turn, can help urge fluid into the second compliant bag 110B.

In an example, a fully expanded fluid volume of the third compliant bag 110C is between 0.05 milliliters and 0.40 milliliters, which can be selected based on an expected or actual intraocular fluid drainage rate from the third compliant bag 110C to leave a sufficient period of time (e.g., between 2 days and 7 days, e.g., 3 days) to be capable to accumulate enough fluid and to promote controlled bleb formation about the reservoir 108, such that any resulting bleb tissue reaction is not too thick to inhibit transfer of intraocular fluid from the reservoir 108 and out to the vasculature to be returned to circulation via the vasculature, as similarly explained above with respect to the first compliant bag 110A.

Although the above description has focused on an example that can include three compliant bags 110A-C, placed in series with each other to provide the reservoir 108, other examples can include a single compliant bag 110A or more than three compliant bags 110 used to provide the reservoir, some of these compliant bags 110 can be in series with others of these compliant bags 110, some of the compliant bags 110 can be in parallel with others of these compliant bags 110, and some of these compliant bags can be placed in a series-parallel combination with others of these compliant bags 110.

Although the above description has focused on a valved or unvalved output port 114 from a compliant bag 110 feeding a downstream compliant bag 110, such output ports 114 can additionally include a valved or unvalved output port 114 from within the compliant bag 110 and out of the reservoir 108 and into a physiological region of the subject that is located outside of the reservoir 108. Although the above description has focused (for illustrative brevity) on a valve 116 of an output port 114 (or located in the tube shunt 108, such as at its distal end 104) feeding another compliant bag 110 chamber of the reservoir 108, it is understood that the valve-opening valve differential pressure for opening a valve associated with the port will be defined by an upstream pressure (just upstream of the valve) and a downstream pressure (just downstream of the valve). For a port and valve that opens into a secondary compliant bag 110, this downstream pressure is the fluid pressure present in the downstream bag. For a port and valve that opens from the reservoir 108 into a physiological space, the valve opening pressure will be defined by a differential between the upstream pressure, and the downstream pressure in the physiological space.

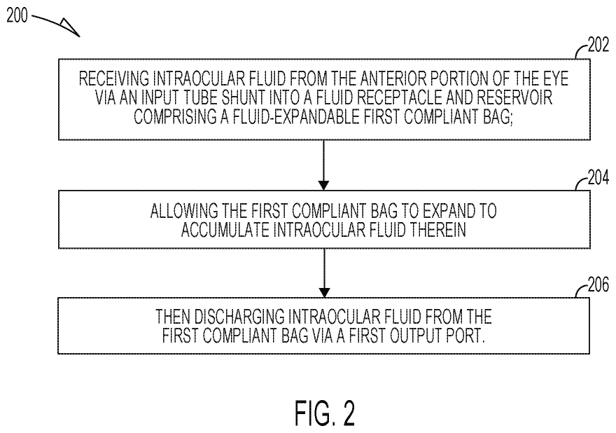

FIG. 2 is a flow chart illustrating generally portions of a method 200 of using a device, such as shown in FIG. 1, for draining intraocular fluid from an anterior portion of an eye of a subject. At 202, the method 200 can include receiving intraocular fluid from the anterior portion of the eye via an input tube shunt into a fluid receptacle and reservoir comprising a fluid-expandable first compliant bag. At 204, the method 200 can include allowing the first compliant bag to expand to accumulate intraocular fluid therein. At 206, the method 200 can include then discharging intraocular fluid from the first compliant bag via a first output port. As explained above, such fluid discharge can be (2) from within a compliant bag 110 included in the reservoir 108 to within another compliant bag 110 that is also included within the reservoir 108, or (2) from within a compliant bag 110 that is included within the reservoir 108 to a physiological location within the subject that is outside of the reservoir 108. Such fluid discharge can be controlled by a valve that can be included within the port, which can automatically open (e.g., without requiring end-user intervention) when a valve-opening differential pressure across the valve is attained.

Thus, when sufficient pressure gradient exists, one-way aqueous flow will occur to downstream bags/chambers (or to a location outside of the reservoir 108) which can also have lateral flow conduits, apertures, or ports on the fluid-expandable compliant bag to allow for externalization of accumulated fluid from the bag/device, such as out from the reservoir 108 and into the extraocular/subconjunctival space.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An intraocular fluid drainage device comprising:
an input tube shunt, providing a fluid conduit between a distal end of the tube shunt, which is configured for insertion into an anterior chamber of an eye of a subject, and a proximal end of the tube shunt; and
a fluid receptacle and reservoir comprising a fluid-expandable first compliant bag, the first compliant bag including a first input port coupled to the proximal end of the tube shunt to receive intraocular fluid therefrom, and the first compliant bag including a first output port from which to discharge intraocular fluid.

2. The device of claim 1, in which the first output port includes a first valve configured to open to discharge intraocular fluid from the first compliant bag when a first fluid pressure within the first compliant bag has reached a specified first threshold pressure.

3. The device of claim 2, wherein the first valve is configured to open to discharge intraocular fluid from the first compliant bag when the first fluid pressure within the first compliant bag has reached a specified first threshold pressure of at least 15 mm Hg.

4. The device of claim 1, in which a fully expanded fluid volume of the first compliant bag is between 0.05 milliliters and 0.40 milliliters.

5. The device of claim 1, wherein the first output port from the first compliant bag provides a fluid pathway out from the fluid receptacle and reservoir into a physiological region of the subject.

6. The device of claim 1, wherein the first output port from the first compliant bag provides a fluid pathway into a fluid-expandable second compliant bag, also included within the fluid receptacle and reservoir, the second compliant bag including an input port included with or coupled to the first output port of the first compliant bag to receive intraocular fluid therefrom, and the second compliant bag including a second output port from which to discharge intraocular fluid.

7. The device of claim 6, in which the second output port includes a second valve configured to open to discharge intraocular fluid from the second compliant bag when a second fluid pressure within the second compliant bag has reached a specified second threshold pressure.

8. The device of claim 7, wherein the second valve is configured to open to discharge intraocular fluid from the second compliant bag when the second fluid pressure within the second compliant bag has reached a specified second threshold pressure of at least 12 mm Hg.

9. The device of claim 6, in which a fully expanded fluid volume of the second compliant bag is between 0.05 milliliters and 0.40 milliliters.

10. The device of claim 6, wherein the second output port from the second compliant bag provides a fluid pathway out from the fluid receptacle and reservoir into a physiological region of the subject.

11. The device of claim 6, where the second output port from the second compliant bag provides a fluid pathway into a fluid-expandable third compliant bag, included within the fluid receptacle and reservoir, the third compliant bag including a third input port included with or coupled to the second output port of the second compliant bag to receive intraocular fluid therefrom, and the third compliant bag including a third output port from which to discharge intraocular fluid.

12. The device of claim 11, in which the third output port includes a third valve configured to open to discharge intraocular fluid from the third compliant bag when a third fluid pressure within the third compliant bag has reached a specified third threshold pressure.

13. The device of claim 12, wherein the third valve is configured to open to discharge intraocular fluid from the third compliant bag when the third fluid pressure within the third compliant bag has reached a specified third threshold pressure of at least 8 mm Hg.

14. The device of claim 11, in which a fully expanded fluid volume of the third compliant bag is between 0.05 milliliters and 0.40 milliliters.

15. The device of claim 11, wherein third first output port from the third compliant bag provides a fluid pathway out from the fluid receptacle and reservoir into a physiological region of the subject.

16. The device of claim 1, wherein the first compliant bag is constructed from a fluid-impervious non-porous material into which the input port and output port are defined to limit fluid flow with respect to the first compliant bag.

17. The device of claim 1, in which the first output port includes a one-way valve.

18. The device of claim 17, in which the first output port includes a duckbill valve.

19. The device of claim 1, in which the first compliant bag includes an expanded fluid volume capable of receiving and accumulating intraocular fluid at 15 mm Hg for a period of time of at least 3 days before discharging intraocular fluid via the first output port to control a bleb formation tissue reaction.

20. The device of claim 1, wherein the first input port includes a one-way valve configured to automatically open when a differential fluid pressure of at least 15 mm Hg is present across from the one-way valve on the first input port.

21. A method of draining intraocular fluid from an anterior portion of an eye of a subject, the method comprising:
receiving intraocular fluid from the anterior portion of the eye via an input tube shunt into a fluid receptacle and reservoir comprising a fluid-expandable first compliant bag;
allowing the first compliant bag to expand to accumulate intraocular fluid therein; and
then discharging intraocular fluid from the first compliant bag via a first output port.

22. The method of claim 21, wherein allowing the first compliant bag to expand to accumulate intraocular fluid therein and then discharging intraocular fluid from the first compliant bag via a first output port, includes:
allowing fluid accumulation for at least a specified first period of time before discharging, wherein the first period of time is specified to control bleb formation about the first compliant bag.

23. The method of claim 21, wherein allowing fluid accumulation is in response to a fluid pressure exceeding a specified first threshold pressure value that is present across the input port.

24. The method of claim 21, wherein discharging intraocular fluid is in response to a fluid pressure exceeding a specified second threshold pressure value that is present in the first compliant bag across the output port.
